(19) United States
(12) Patent Application Publication
(10) Pub. No.: US 2005/0152994 A1
(43) Pub. Date: Jul. 14, 2005

(54) Composition and process for coloring and preserving wood

(76) Inventors: Robert M. Leach, Grand Island, NY (US); Jun Zhang, Getzville, NY (US)

Correspondence Address: HODGSON RUSS LLP, ONE M & T PLAZA, SUITE 2000, BUFFALO, NY, 14203-2391, US

(21) Appl. No.: 11/006,834

(22) Filed: Dec. 8, 2004

Related U.S. Application Data
(60) Provisional application No. 60/527,808, filed on Dec. 8, 2003.

(51) Int. Cl. A61K 33/22

(52) U.S. Cl. 424/719; 424/659

(57) ABSTRACT

The present invention provides compositions which can be used to preserve and color wood, as well as methods of using the solution to both preserve and color wood. The composition is an alkaline solution which comprises one or more metal compounds or metal complexes and one or more cationic dyes

This application claims priority to provisional application Ser. No. 60/527,808, filed Dec. 8, 2003, the disclosure of which is incorporated herein by reference.

### FIELD OF THE INVENTION

This invention relates to a composition and method for both coloring cellulosic products such as wood and preserving wood against various destructive organisms or environmental agents responsible for rot and decay. More particularly, this invention relates to a composition and method whereby the coloring and preserving of the wood may be accomplished simultaneously, in a single application, or sequentially, in two separate applications which may be performed in either order.

### BACKGROUND OF THE INVENTION

It has long been desirable to produce wood products that are artistically and aesthetically acceptable to the public, yet preserved from the destructive agencies of wood, such as fungi and insects. Wood that is colored and preserved is desirable for the home, and is used in the siding, fencing, and decking industry. Unfortunately, many of the preservative solutions used to preserve wood from wood destroying organisms impart their own color to the wood. In many cases the coloration imparted by the preserving solution is undesirable for one reason or the other.

One technique currently used to color wood is to paint the wood with an oil base paint or pigmented coating. Depending on the type of preservative used, some of the coatings will not adhere to the wood, resulting in blistering or flaking in a short period of time. Conventional petroleum based preservatives, such as creosote or pentachlorophenol pose problems because of oily films left on the treated wood, which may require several months to season, and even after such seasoning, it still may be difficult for paint or stain to penetrate the oily residue left behind. Water based preservatives are more desirable than conventional petroleum based preservatives because the wood is not coated with a film and can be readily painted or stained after treatment. However, coloration is only imparted at the surface of the wood with these techniques, resulting in limited permanence to weathering.

Alternatively, the wood may be precoated with a coloring agent such as an oil base or latex paint followed by treatment with the wood preservative. Many of the oil base paints or latex paints will form a coating on the wood which reduces or eliminates the penetration of the preservative into the wood. In many cases, the coatings and preservatives are incompatible with one another.

Many conventional processes are available for coloring and staining wood, while several processes are available to preserve wood from the various wood destroying organisms.

Yet very few satisfactory methods are available to color and preserve the wood simultaneously. The processes that are available are inconvenient because they are multi-stepped operations. Furthermore, many conventional processes provide only a surface coloration which will scratch or wear away and require additional treatment or servicing for exposure to long term weathering.

In order to color wood to enhance the aesthetic appearance of wood, and yet preserve wood against deterioration, a colorant system must be found that is compatible with the wood preservative system. Many conventional wood preserving compositions contain alkaline metal complexes, such as copper amine complexes. The high pH and the cationic nature of the metal complexes limit the use of many dyes in solution with alkaline metal complexes. Dyes such as acid dyes, direct dyes and reactive dyes, generally cannot be used with aqueous solutions of copper amine complexes due to the high pH and cationic nature of the solutions. When mixed with the alkaline metal complex based preservative solutions in an aqueous solution, these dyes either degrade or precipitate.

In view of the many shortcomings applicable to the current methods of coloring and preserving wood, it is desirable to have a process which is not only capable of coloring and preserving the wood by a single operation, but also results in the penetration of the color into the wood to provide for long term application and stability. This need is solved by the subject matter disclosed herein.

### SUMMARY OF THE INVENTION

We have found that cationic dyes can be stable in the presence of alkaline wood preservative solutions which are based on metal complexes or metal compounds.

It is the object of this invention to provide compositions and methods for preserving wood from deterioration by decay and insects, which may also simultaneously color the wood in a single application.

A further object of this invention is to provide a method of impregnating the color beneath the surface of the wood to provide for long term application.

Still another object of this invention is to provide a method for imparting color to wood which results in the appearance of uniform color on the surface of the wood.

In accordance with the present invention there is provided a composition for preserving and coloring wood. The composition comprises an alkaline preservative solution which is based on metal complexes or metal compounds. It further comprises one or more cationic dyes which are substantially unprecipitated in the presence of the alkaline metal complexes or metal compounds, such that the composition can be used to simultaneously preserve and color wood. The cationic dyes include: derivatives of diphenylmethane; triphenylmethane or acridine; thiazine, oxazine, or azine dyes; xanthene basic dyes, basic dyes containing azo groups, or basic dyes containing a pendant cation, a delocalized charge, or a heterocylic ring which contains a quaternary nitrogen atom. Depending upon the specific cationic dyes used, the color of the solutions and treated wood vary from red to brown or somewhere between.

Cationic dyes are known in the coloring industry. However, the use of these compounds in combination with alkaline metal complex based preservative compositions to impart wood coloring capability directly to the preservative composition, is not known. An advantage of using cationic dyes is that compared to other classes of dyes, cationic dyes have a higher affinity for wood and form a strong chemical bonding with wood. This results in rapid color development and negligible wash-off of colorants when exposed outdoors.

When wood is treated with the preservative composition of the present invention according to conventional wood treatment cycles employing for example, the Full Cell or Empty Cell process or some combination thereof, or by dip or spray treatment, it is believed that both the preservative and the dye are carried beneath the surface of the wood to thereby impart long lasting color to the wood. In this manner the color and preservative are applied in a single step operation.

### DETAILED DESCRIPTION OF THE INVENTION

The present invention provides compositions and methods for preserving and coloring wood and wood products. The composition comprises a preservative solution comprising metal compounds and/or metal complexes, such as, for example, copper, silver or zinc complexes, and one or more cationic dyes. In a preferred embodiment, the metal complexes are copper complexes.

Cationic dyes are so called because they dissociate upon dissolution in water, with the cation having the color properties of a dye. The cationic dyes used herein include: derivatives of diphenylmethane; triphenylmethane or acridine; thiazine, oxazine, or azine dyes; xanthene basic dyes, basic dyes containing azo groups, and basic dyes containing a pendant cation, a delocalized charge, or a heterocylic ring which contains a quaternary nitrogen atom.

The preferred cationic dyes are the basic dyes containing monoazo or azo groups, methine and thiazine based dyes. These dyes have been discovered to demonstrate the greatest stability in alkaline metal compound or metal complex solutions.

Examples of derivatives of diphenylmethane are: 2-[4-(dimethylamino)phenyl]-3,6-dimethyl-chloride; 1,1-bis(p-dimethylaminophenyl)methylenimine hydrochloride, as well as other compounds.

Examples of derivatives of triphenylmethane include: Methanaminium N-[4-[[4-(dimethylamino)phenyl]phenylmethylene]-2,5-cyclohexadien-1-ylidene]-N-methyl-, chloride; 4-((4-aminophenyl)(4-imino-2,5-cyclohexadien-1-ylidene)methyl)-2-methyl-benzenamine; 4,4′-((4-imino-2,5-cyclohexadien-1-ylidene)methylene)dianiline monohydrochloride.

Examples of thiazine dyes include: 3,7-Bis(dimethylamino)phenothiazin-5-ium chloride; 7-(Dimethylamino)-6-nitro-3H-phenothiazin-3-ylidene)dimethylammonium chloride.

Examples of methine or polymethine dyes include: 2-(((4-Methoxyphenyl)methylhydrazono)methyl)-1,3,3-trimethyl-3H-indolium methyl sulphate; 2-(2-(4-((2-Chloroethyl)methylamino)phenyl)vinyl)-1,3,3-trimethyl-3H-indolium chloride.

Examples of oxazine dyes include: 9-(Dimethylamino)benzo(alpha)phenoxazin-7-ium chloride; 5H-Benzo(a)phenazoxazine, 9-diethylamino-5-imino-, hydrochloride.

Examples of azine dyes include: 3-Amino-7-dimethylamino-2-methylphenazine hydrochloride;

Examples of xanthene dyes include: N-(9-(2-Carboxyphenyl)-6-(diethylamino)-3H-xanthen-3-ylidene)-N-e-thylethanaminium chloride; 9-(2-(Ethoxycarbonyl)phenyl)-3,6-bis(ethylamino)-2,7-dimethylxanthylium chloride.

Examples of derivatives of acridine include: 3,6-Acridinediamine, N,N,N′,N′-tetramethyl-, monohydrochloride compd. with zinc chloride;

Examples of basic dyes containing azo groups include: 1,3-Benzenediamine, 4,4′-(1,3-phenylenebis(azo))bis-, dihydrochloride; 3-Methyl-2-((1-methyl-2-phenyl-1H-indol-3-yl)azo)thiazolium chloride; (2-((4-((2-Chloro-4-nitrophenyl)azo)phenyl)ethylamino)ethyl)trimethylammonium methyl sulphate; 2-(((1,3-Dihydro-1,3-dimethyl-2H-benzimidazol-2-ylidene)methyl)azo)-3-methylbenzothiazolium methyl sulphate.

Examples of basic dyes containing a pendant cation include: (2-((4-((2-Chloro-4-nitrophenyl)azo)phenyl)ethylamino)ethyl)trimethylammonium;

Examples of basic dyes with a delocalized charge include: 4-((2-Chlorophenyl)(4-(ethylimino)-3-methylcyclohexa-2,5-dien-1-ylidene)methyl)-N-ethyl-o-toluidine monohydrochloride; 2-(2-(4-((2-Chloroethyl)ethylamino)-o-tolyl)vinyl)-1,3,3-trimethyl-3H-indolium chloride.

The wood preserving/coloring solutions of the present invention are generally alkaline, and the alkalinity may be due, al least in part, to the alkaline nature of the metal complexes, if the solution contains such complexes. However, the solution may be alkaline for other reasons, such as the case with a metal compound which is in solution with an alkaline cobiocide. The solution should have a pH of between 7.0 and 13.0, but preferably in the range of from 8.5 to 10.0.

In addition to a cationic dye, the wood preservative solution of the present invention also comprises an alkaline metal complex and or a metal compound. Suitable metals include copper, arsenic, zinc, silver, cadmium, nickel, bismuth, lead and chromium, with copper being preferred.

It is preferred to have a metal complex or metal compound concentration in the range of from 0.001% to 10% by weight of the solution.

If metal complexes are desired in the wood preservative composition of the present invention, a wide variety of complexing agents can be used to make them. Examples of appropriate complexing agents which can be used include nitrogen-containing complexing agents such as ammonium compounds, quaternary ammonium compounds and their salts; amines (including ammonia) and alkanolamines, such as, for example monoethanolamine (MEA); and alkane- and alkene-bearing amine compounds such as, for example ethylene diamine (EDA), ethylene diamine tetraacetate (EDTA), and diethylene triamine (DETA); polyamines and combinations thereof.

The metal may also be present as a mixture in which other metals. If the metal is copper, other metals such as, for example, arsenic, chromium, silver, bismuth, lead nickel or cadmium can be present, either in their elemental forms, or as binary or other compounds.

The metal compounds which can be used in the present invention preferably have appreciable water solubility such that they can form alkaline aqueous solutions.

Other components may be present in the solution, as long as they do not lead to clouding of the dye solution such that it can no longer be used to color wood. Preferred are components, such as tebuconazole which have biocidal properties and can be used as cobiocides, yet do not result in appreciable precipitation of the dye. In general, cobiocides which can be used include azoles, boric acid, borate compounds, fluoride compounds, quaternary ammonium compounds, polybetains and combinations thereof.

Suitable complexes can be obtained commercially as concentrates, and non-limiting examples of commercially available alkaline metal complex preservative concentrates that can be used with the cationic dyes of this invention are:

(1) Ammoniacal copper arsenate: containing about 45.0-55.0% copper as copper oxide and about 45.0-55.0% arsenic as arsenic pentaoxide.

(2) Ammoniacal copper zinc arsenate: containing about 45.0-55.0% copper as copper oxide, about 22.5-27.5% zinc as zinc oxide and about 22.5-27.5% arsenic as arsenic oxide.

(3) ACQ-type A: containing about 45.0-55.0% copper as copper oxide and about 45.0-55.0% quaternary ammonium compounds.

(4) ACQ-type B: containing about 62.0-71.0% copper as copper oxide and about 29.0-38.0% quaternary ammonium compounds.

(5) ACQ-type C: containing about 62.0-71.0% copper as copper oxide and about 29.0-38.0% quaternary ammonium compounds.

(6) ACQ-type D: containing about 62.0-71.0% copper as copper oxide and about 29.0-38.0% quaternary ammonium compounds.

(7) Cu-HDO: containing about 58-65% copper as copper oxide, about 10-18% % bis-(N-cyclohexyldiazeniumdioxide) (HDO) and about 17-32% boron as boric acid.

(8) Ammoniacal copper citrate: containing about 59.0-68.0% copper as copper oxide and about 32.0-41.0% citric acid.

(9) Copper Azole-typeA (CBA-A): containing about 44.0-54.0% copper, about 44.0-54.0% boron as boric acid and about 1.8-2.8% azole as tebuconazole.

(10) Copper Azole-typeB(CA-B): containing about 95.4-96.8% copper and 3.2-4.6% azole as tebuconazole.

In the above exemplary formulations, metal components are expressed in their weight percent equivalents as oxides, as is frequently done in the art.

In the wood preservative solutions of the present invention, it is preferable that the metal be present in the solution applied to wood in amounts in the range of from 0.005 to 10 weight percent of the of the solution (the convention of expressing metal content in terms of metal oxides is not used in this range expression). This range includes all of the metals in the solution, regardless of whether they are present as complexes, compounds, or both.

To prepare the composition of this invention, the cationic dyes may be prepared as an aqueous dye paste containing about 40 to 60% solids. The mixture is then combined with a solution of the water soluble preservative to obtain a final dye concentration ranging from 0.001% to 5.0% by weight of the solution, depending upon the concentration of the metal present in the preservative solution. Concentrations above 5.0% may also be used, however, the preferred final concentration of the dye in solution is about 0.1% to 2% by weight. Generally, concentrations ranging from 0.01% to 10.0% may be used.

Wood preservative solutions according to the present invention allow the dye to be present in solution with alkaline metal complexes without undergoing significant degradation or precipitation. It is not unusual for the cationic dye in such solutions to be useful in imparting color to wood and remain unprecipitated for periods of time exceeding 10 days. By “substantially unprecipitated,” it is meant that the dye solution remains clear enough such that it can be used to color wood.

Temperature and pressure parameters are not critical for applying the dye solutions of the present invention to wood. A fairly wide temperature range may be employed, with the upper limit at about 150° F. The lower limit should be sufficiently warm to prevent the solution from freezing.

A wide range of useful colors can be imparted to wood using the process of the present invention. The color of wood treated with the preservative solutions described herein can be brown, red or other shades, depending upon the particular combination of cationic dyes, and their concentration. The particular metal or combination of metals present in the preservative solution will also influence the final coloration imparted to the wood.

By applying the composition of the present invention to wood, it can be simultaneously preserved and colored with the application of a single solution. The treating solution may be applied to wood by dipping, soaking, spraying, brushing, or any other means well known in the art. In a preferred embodiment, vacuum and/or pressure techniques are used to impregnate the wood in accord with this invention including the standard processes, such as the “Empty Cell” process, the “Modified Full Cell” process and the “Full Cell” process, and any other vacuum and/or pressure processes which are well known to those skilled in the art.

The standard processes are defined as described in AWPA Standard C1-03 “All Timber Products—Preservative Treatment by Pressure Processes”. In the “Empty Cell” process, prior to the introduction of preservative, materials are subjected to atmospheric air pressure (Lowry) or to higher air pressures (Rueping) of the necessary intensity and duration. In the “Modified Full Cell”, prior to introduction of preservative, materials are subjected to a vacuum of less than 77 kPa (22 inch Hg) (sea level equivalent). A final vacuum of not less than 77 kPa (22 inch Hg) (sea level equivalent) should be used. In the “Full Cell Process”, prior to introduction of preservative or during any period of condition prior to treatment, materials are subjected to a vacuum of not less than 77 kPa (22 inch Hg). A final vacuum of not less than 77 kPa (22 inch Hg) is used.

Before impregnating timber with any wood treating solution it is preferable to season it first until at least all the free water has been removed from the cell spaces. This stage of seasoning represents a moisture content of about 25-30%, varying slightly with different species. There are two reasons for this: first, it is difficult to inject another liquid into wood containing which contains significant amounts of water, and second, splits developing as the result of the subsequent drying of the timber would likely expose untreated timber.

It is also desirable to carry out cutting, machining and boring, etc., of the timber before treatment is applied, as all these operations, if carried out after treatment, would expose untreated wood. Where these operations cannot be performed until after treatment all exposed untreated timber should be given a liberal application of treating solution, and holes preferably treated with a pressure bolt-hole treater.

It is preferable to color and preserve the wood simultaneously, however, in certain instances it may be desirable to treat and color the wood in two stages. Without departing from the teachings of this invention the wood may first be treated with a solution containing the alkaline metal complex, and then contacted with a solution containing the cationic dye. It is also possible to apply the coloring agent to the wood initially, followed by the application of the preservative solution. It is preferable that the concentrations in each solution conform to the concentrations of alkaline metal complex and cationic dye given above for the two component solution. As with application of a two component solution, the application process can be carried out using known conventional vacuum and/or pressure techniques. The two step application is particularly useful in wood treatment processes in which solutions of dye or preservative which have been exposed to the wood but not absorbed are reused. With the present invention, little, if any, precipitate is formed in the recirculated fluid after it has been exposed to wood even though the fluid may have been contaminated such that both dye and preservative are together in solution.

The following examples will serve to further illustrate the invention.

### EXAMPLE 1

Southern Yellow Pine, (measuring 2″×6″×4′) was simultaneously colored and preserved by the Full Cell treatment using a 1.1% ACQ-B solution containing 0.73% copper oxide and 0.37% quaternary ammonium compounds, and 0.12% cationic dye mixture of 1,1-bis(p-dimethylaminophenyl)methylenimine hydrochloride and 4,4′-((4-imino-2,5-cyclohexadien-1-ylidene)methylene)dianiline monohydrochloride. The wood was initially placed under a vacuum of 30″ Hg for 30 minutes followed by the addition of the treating solution. The system was then pressurized for 30 minutes at a pressure of 110 lbs. per square inch. The resulting wood, when dried, was colored brown and was also protected against wood destroying organisms.

### EXAMPLE 2

Douglas-fir blocks (1½″×2″×6″) were simultaneously colored and preserved utilizing the Lowry Empty Cell process using a 1.5% ACQ-D treating solution containing 1.0% copper oxide, and 0.5% quaternary ammonium compounds, and 0.22% cationic dye mixture of 3-Amino-7-dimethylamino-2-methylphenazine hydrochloride and 1,3-Benzenediamine, 4,4′-(1,3-phenylenebis(azo))bis-, dihydrochloride. The resulting wood was air dried to a 20% moisture content and was colored a light brown.

### EXAMPLE 3

0.45 grams of 1,3-Benzenediamine, 4,4′-(1,3-phenylenebis(azo))bis-, dihydrochloride were added to 100 grams of water. This solution was then added to 900 grams of a 1.1% preservative solution containing 1.6% copper as copper oxide and 0.8% dimethyldidecylammonium carbonate. After 1 1/2 hours of reaction, Western Hemlock was treated using the Full Cell process. The resulting wood was oven dried at 120° F. and was colored a dark brown throughout.

### EXAMPLE 4

Southern Yellow Pine blocks (½″×2″×6″) were simultaneously colored and preserved using the Full Cell treatment with a solution containing 3-Methyl-2-((1-methyl-2-phenyl-1H-indol-3-yl)azo)thiazolium chloride, 2-(((4-Methoxyphenyl) methylhydrazono)methyl)-1,3,3-trimethyl-3H-indolium methyl sulphate and 9-(Dimethylamino)benzo(alpha)phenoxazin-7-ium chloride in a 1.5% ACQ-type D solution. The Southern Yellow Pine blocks were placed in a cylinder and a vacuum of 30″ Hg applied for 15 minutes, the treating solution was then added to the cylinder and the system pressurized to 100 lbs. per square inch for 30 minutes. The resulting wood, when dried, was colored a light brown and was also protected against wood destroying organisms.

### EXAMPLE 5

Southern Yellow Pine blocks were colored a light brown color with a solution containing a mixture of 1,1-bis(p-dimethylaminophenyl)methylenimine hydrochloride, 4,4′-((4-imino-2,5-cyclohexadien-1-ylidene)methylene)dianiline monohydrochloride and 3,7-Bis(dimethylamino)phenothiazin-5-ium chloride in a 1.5% ACQ-Type D solution using the Full Cell process.

### EXAMPLE 6

Southern Yellow Pine blocks were colored a light brown color with a two-step process. Step I involved the treatment of wood with 1.1% ACQ-Type D solution using the Full Cell process, followed by Step II treatment with a solution containing a mixture of 1,1-bis(p-dimethylaminophenyl)methylenimine hydrochloride, 4,4′-((4-imino-2,5-cyclohexadien-1-ylidene)methylene)dianiline monohydrochloride and 3,7-Bis(dimethylamino)phenothiazin-5-ium chloride.

A variety of cellulosic products such as wood, paper, textiles, cotton and the like can be colored and preserved in accordance with this invention including hard and/or soft woods. In general, wood may thus be simultaneously colored and preserved provided it is capable of withstanding the wood treatment processes.

Wood colored and preserved according to the method of this invention resists weathering and has many uses in the construction industry. Patio and pool decks, wood siding and beams, fence posts, garden ties and poles for outdoor or indoor use are just a few of the possible products which may incorporate wood treated according to the method described herein.

It is to be fully understood that all of the foregoing examples are intended to be merely illustrative and not to be construed or interpreted as being restrictive or otherwise limiting of the present invention.

1. A method for preserving wood against wood destroying organisms and coloring the wood, said method comprising contacting the wood concurrently or sequentially with:
(a) an alkaline solution comprising a metal complex or a metal compound; and
(b) a cationic dye.

2. The method of claim 1, wherein the alkaline solution (a) further comprises a cobiocide.

3. The method of claim 1 wherein the metal is selected from the group consisting of copper, nickel, arsenic, zinc, silver, cadmium, bismuth, lead and chromium.

4. The method of claim 3, wherein the metal is copper.

5. The method of claim 4 wherein the metal complex comprises a nitrogen containing complexing selected from the group consisting of ammonia, ammonium compounds, alkanolamine, polyamines, quaternary ammonium compounds or combinations thereof.

6. The method of claim 5, wherein the alkanolamine is monoethanolamine.

7. The method of claim 1, wherein the alkaline solution (a) further comprises arsenic, zinc, citrate, polybetains, or bis-(N-cyclohexyldiazeniumdioxide).

8. The method of claim 1, wherein contacting the wood concurrently with (a) and (b) comprises contacting the wood with a solution comprising (a) and (b).

9. The method of claim 2, wherein the cobiocide is selected from the group consisting of boric acid, a borate, a fluoride, an azole, a quaternary ammonium compound, a polybetain and combinations thereof.

10. The method of claim 1 wherein the cationic dye is selected from the group consisting of derivatives of diphenylmethane, derivatives of triphenylmethane, thiazine dyes, oxazine dyes, azines, xanthene basic dyes, derivatives of acridine, basic dyes having azo groups, and basic dyes having a pendant cation, a delocalized charge or a heterocylic ring containing a quaternary nitrogen atom.

11. The method of claim 10 wherein the concentration of said cationic dye is in the range of from 0.001% to 5.0% by weight of the solution.

12. The method of claim 11 wherein the concentration of the metal complex is in the range of from 0.001% to 10% by weight of the solution.

13. The method of claim 8 wherein the pH of the solution is 7.0 to 13.0.

14. The method of claim 13 wherein the pH of the solution is 8.5 to 10.0.

15. The method of claim 8 wherein the cationic dye is substantially unprecipitated.

16. The method of claim 8 wherein the solution is applied to the wood using a process selected from the group consisting of full cell process, modified full cell process, empty cell process, dipping and spraying.

17. A composition for preserving wood against wood destroying organisms and coloring the wood, which comprises (a) an alkaline solution comprising a metal complex or a metal compound; and (b) one or more cationic dyes.

18. The composition of claim 17 further comprising a cobiocide.

19. The composition of claim 17, wherein the cationic dye is substantially unprecipitated.

20. The composition of claim 17 wherein the metal is selected from the group consisting of copper, arsenic, nickel, zinc, silver, cadmium, bismuth, lead and chromium.

21. The composition of claim 20 wherein the metal is copper.

22. The composition of claim 21, wherein the metal complex comprises a nitrogen containing complexing agent selected from the group consisting of ammonia, ammonium compounds, alkanolamine, polyamines, quaternary ammonium compounds and combinations thereof.

23. The composition of claim 22, wherein the alkanoamine is monoethanolamine.

24. The composition of claim 17, wherein the composition further comprises arsenic, zinc, citrate, polybetains or bis-(N-cyclohexyldiazeniumdioxide)

25. The composition of claim 18, wherein the composition further comprises a cobiocide selected from the group consisting of boric acid, a borate, a fluoride, an azole, a quaternary ammonium compound, a polybetain and combinations thereof.

26. The composition of claim 17 wherein the cationic dye is selected from the group consisting of derivatives of diphenylmethane, derivatives of triphenylmethane, thiazine dyes, oxazine dyes, azines, xanthene basic dyes, derivatives of acridine, basic dyes having azo groups, and basic dyes having a pendant cation, a delocalized charge or a heterocylic ring containing a quaternary nitrogen atom.

27. The composition of claim 26 wherein the concentration of said cationic dye is in the range of from 0.001% to 10.0% by weight of the solution.

28. The composition of claim 27 wherein the concentration of said alkaline metal complex is in the range of from 0.001 to 10% by weight of the solution.

29. The composition of claim 17 wherein the pH of the solution is 7.0 to 13.0.

30. The composition of claim 29 wherein the pH of the solution is 8.5 to 10.0.

31. Wood which has been treated by the process of claim 1.
